(19) United States
(12) Patent Application Publication
(10) Pub. No.: US 2004/0188430 A1
(43) Pub. Date: Sep. 30, 2004

(54) Microbial decontaminator

(76) Inventors: Ghulam Nabi Qazi, Chandigarh (IN); Satish Chander Puri, Chandigarh (IN); Anupama Braroo, Chandigarh (IN); Vijeshwar Verma, Chandigarh (IN); Syed Riyaz Ul-Hassan, Chandigarh (IN); Rajneesh Anand, Chandigarh (IN); Amol P. Bhondekar, Chandigarh (IN); Ashwani Kumar, Chandigarh (IN); Lalit M. Bharadwaj, Chandigarh (IN); R. P. Bajpai, Chandigarh (IN)

Correspondence Address: RATNERPRESTIA, P O BOX 980, VALLEY FORGE, PA, 19482-0980, US

(21) Appl. No.: 10/403,761

(22) Filed: Mar. 31, 2003

(51) Int. Cl. A61L 2/12; H05B 6/64

(52) U.S. Cl. 219/700; 219/679; 422/21

(57) ABSTRACT

A microbial decontaminator device for decontaminating herbal and/or pharmaceutical products, the said decontamination device comprising of a microwave oven provided with two slots, one on a front face and the other on a rear face of the microwave oven, a conveyor belt passing through the microwave oven through the two slots and provided with a driving means for driving the conveyor belt, said driving means being coupled to a computer which controls the driving means and in turn, the conveyor belt, the assembly of the microwave oven, the conveyor belt and the driving means being covered by a shielding cage to prevent leakage of microwave radiation to atmosphere, a feed hopper provided behind the rear face of the microwave oven and the conveyor belt for feeding the product to be decontaminated, and an output chute being provided below the conveyor belt and in front of the front face of the microwave oven for collecting the decontaminated product.

### FIELD OF THE INVENTION

The present invention relates to microbial decontaminator device for microbial decontamination of herbal and/or pharmaceutical products. The device of the present invention is particularly useful to develop, demonstrate and promote microwave irradiation process technology for its diverse application by conducting applied research.

### BACKGROUND ART

Microwave irradiations are used for rapid sterilization in medicine and industries for different proposes such as decontamination of surgical scalpels, medical wastes, food processing and air sterilization.

Microwave electromagnetic radiations having frequency range of 300-3000 GHz, and more particularly having a frequency of 2540 GHz are used in conventional microwave ovens. Absorption of this radiation, in materials containing water, produces friction between water molecules under alternating electrical fields which produces heat and kills the microorganisms.

Medicinal plants generally carry variety of microbial contaminants, which are represented by bacteria, fungi and viruses. Bacterial endospores and fungal spores can be regarded as the two dominating groups of contaminants associated with medicinal plants.

The current practices of harvesting, handling and production, often causes additional contamination and microbial growth. It is not always possible to produce herbal drug raw material free from any kind of harmful microbial infestation, pesticides residues and soil insects etc. Post-harvest disease control will solve the problem of storage transport and marketing consumable herbal products. Today control of post harvest contaminations in Ayurvedic and Herbal products is a challenging technology.

Therefore, it is intended to carry out work on microbial decontamination of medicinal plant material/crude plant material used in the traditional system of medicines at continuous scale, to produce decontaminated medicinal plant material/crude plant material which is accepted by the European Herbal Infusion Association (EHIA) for microbiological threshold tolerance levels.

### OBJECTS OF THE PRESENT INVENTION

The main object of the present invention is to provide a very simple and compact machine with high productivity to elucidate microbiological criteria and methodological aspects which IS useful to be further integrated in modern quality assurance with hygiene parameters set up by modern HACCP (Hazard Analysis and critical Control Point) and ISO.

Another object of the present invention is to provide a machine for establishing criteria for microbial death and irreversible cessation of vital functions such as growth, reproduction, inability to attach and infestation of practical level.

Yet another object of the present invention is to provide a machine that contains the bio-load or bio-burden so as to reflect the desired degree of safety without any changes in the content of specific compounds with particular pharmaceutical and medical relevance.

Still another object of the present invention is to provide a microbial decontaminator for medicinal plants, that host a wide spectrum of microorganisms with various properties with considerable difference regarding qualitative and quantitative aspects.

One more object of the present intention is to provide a machine for the production of decontaminated herbal drugs under simple, systematic and sterilized conditions.

A further object of the present invention is to provide a simple, continuous and compact machine for microbial decontamination.

### SUMMARY OF THE INVENTION

Accordingly, the present invention relates to microbial decontaminator device for microbial decontamination of herbal and/or pharmaceutical products in a continuous fashion using a microwave oven.

### DETAILED DESCRIPTION OF THE INVENTION

The present invention provides a microbial decontaminator device for decontaminating herbal and/or pharmaceutical products, the said decontamination device comprising of:

a microwave oven provided with two slots, one on a front face and the other on a rear face of the microwave oven;

a conveyor belt passing through the microwave oven through the two slots and provided with a driving means for driving the conveyor belt;

said driving means being coupled to a computer which controls the driving means and in turn, the conveyor belt;

the assembly of the microwave oven, the conveyor belt and the driving means being covered by a shielding cage to prevent leakage of microwave radiation to atmosphere;

a feed hopper provided behind the rear face of the microwave oven and the conveyor belt for feeding the product to be decontaminated, and

an output chute being provided below the conveyor belt and in front of the front face of the microwave oven for collecting the decontaminated product.

In an embodiment of the present invention, the microwave oven is a commercially available microwave oven operating at a power level in the range of 800 to 900 watts.

In another embodiment of the present invention, a knob for controlling the status of the microwave oven and the operating power of the microwave oven is provided outside the shielding cage.

In yet another embodiment of the present invention, the slots provided on the microwave oven are enough to let the conveyor to pass through.

In still another embodiment of the present invention, the capacity of the microwave oven is 28 liters.

In one more embodiment of the present invention, the conveyor belt is constructed of Teflon.

In one another embodiment of the present invention, the conveyor belt passes over two rollers of same size.

In an embodiment of the present invention, the rollers are coupled to the driving means through a coupling mechanism for driving the rollers.

In another embodiment of the present invention, the rollers are provided with bearings for coupling to the driving means.

In yet another embodiment of the present invention, the driving means is a motor.

In still another embodiment of the present invention, the motor is coupled to the computer using a parallel port or a LPTI printer port.

In one more embodiment of the present invention, the time period for which the herbal or the pharmaceutical product is exposed to the microwave radiation is controlled by the computer.

In one another embodiment of the present invention, the shielding cage forms a Faraday's cage thereby preventing leakage of microwave radiation to the atmosphere.

In an embodiment of the present invention, the Faraday's cage is made up of M.S. Mesh and acrylic sheets.

In another embodiment of the present invention, the Faraday's cage is made up of M.S. Mesh having 0.1 mm diameter holes and 6 mm thick acrylic sheet.

In yet another embodiment of the present invention, the feeding hopper is provided such that a feeding end of the hopper is above the shielding cage and a discharge end is inside the shielding cage.

In a further embodiment of the present invention, the output chute is provided such that an output-collecting end is inside the shielding cage while an output discharge end is outside the shielding cage.

The following paragraphs illustrate the invention by way of examples of principles of the invention and describe several embodiment adaptations, variations, alternatives and uses of the invention including what we presently believe the best mode carrying out invention. This description will clearly enable one skilled in the art to make and use of the invention. The following examples are given by the way of illustrations of the present invention should not be construed to limit the scope of the present invention in any manner.

### BRIEF DESCRIPTION OF THE ACCOMPANYING DRAWINGS

In the drawings accompanying the specification,

FIG. 1 represents the front view of the decontamination device of the present invention without the shielding covers.

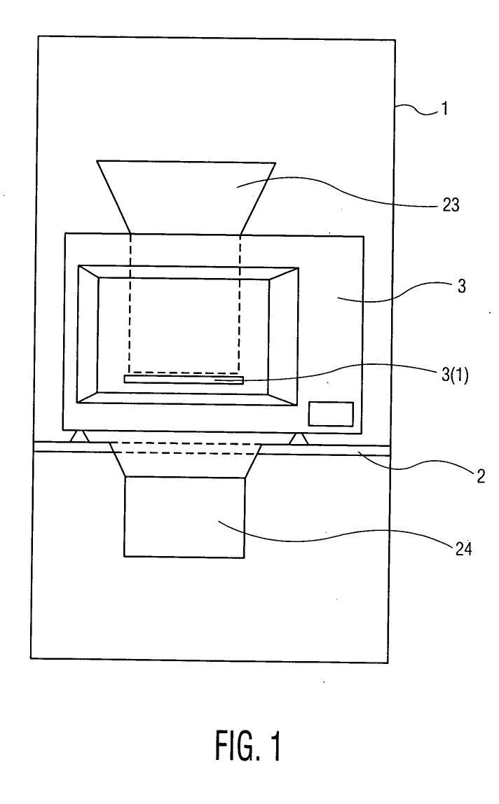

FIG. 2 represents the top view of the decontamination device of the present invention without the shielding covers.

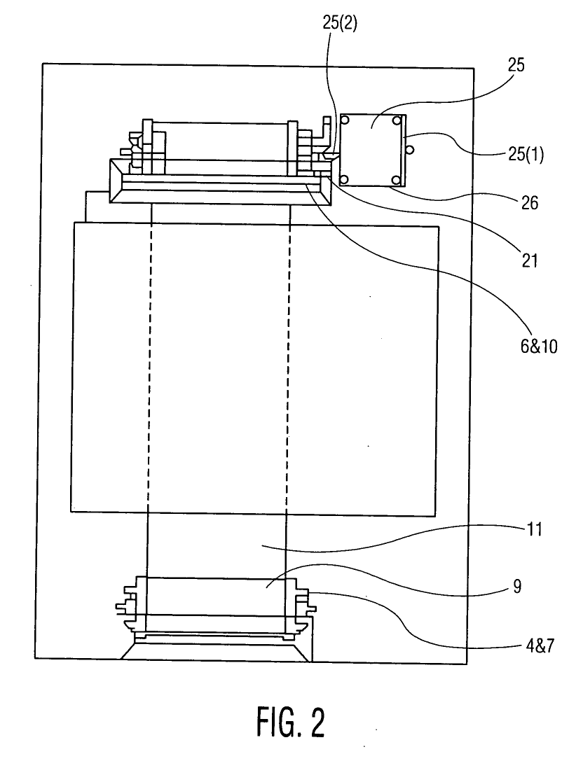

FIG. 3 represents the side view of the decontamination device of the present invention without the shielding covers.

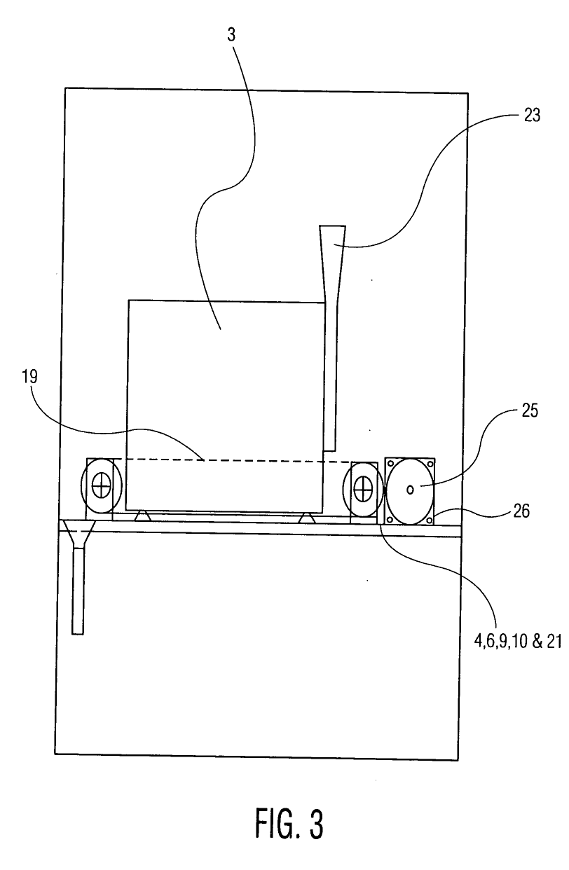

FIG. 4 represents the top and the side view of the shielding covers.

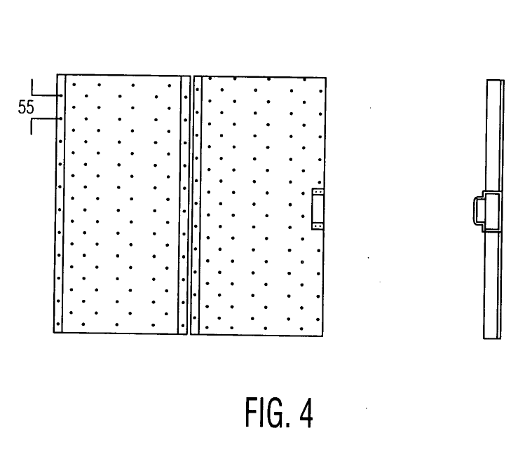

### EXAMPLE 1

### The Structure and Working on the Machine

The equipment consists of a microwave oven (3) having a capacity of 28 liters with one slot (3(1)) each cut out in its front side and the rear panel to pass a conveyor belt (11) through it. The dimension of the conveyor belt is of 1380×200 mm. The slot is just enough to let the conveyor belt to pass through it along with the sample. There are two aluminium rollers (9) of the same dimension over which the conveyor belt rolls and passes through the microwave oven. The rollers have bearings fitted in that are used for coupling the roller to a motor (25) using a gear. A computer (not shown in the figure) coupled to the motor controls the motion of the conveyor belt. The motor is interfaced to the computer using the parallel port or LPTI printer port. A Visual C++based software runs on the computer and controls the motion of the motion of the conveyor belt.

The sample to be decontaminated is fed onto the conveyor belt from a feeder hopper (23) located at the rear of the microwave oven. The sample fed from the rear side is collected from the front side after decontamination using a material receiving hood (24). The sample is exposed to microwave radiations for a particular period of time and the amount of exposure of the product is controlled by:

1. controlling the time period for which sample is exposed to the microwave radiation;

2. controlling the power of the microwave radiation to which the sample is exposed.

The amount of decontamination required is decided by the chemical composition of the sample. To avoid leakage of microwave radiation, the device of the present invention is shielded using M.S. Mesh and acrylic sheets.

When the sample is subjected to microwaves, the harmful microbes present in it are heated up on account of motion due to microwave energy and are killed in the process hence leaving the sample decontaminated.

The sample is first fed on the conveyor belt in sterilized Kraft paper envelops. The sample should not be left stationary and it has to be kept moving while decontamination is going on. The motor and the conveyor belt is started using the software from the computer. The software also controls the exposure time. The conveyor belt stops automatically after moving for the stipulated time. There is no need to switch off the conveyor belt manually.

Powdered drugs (Hypericum perforatum), sieved through BS 30 mesh were taken. 10 gms of the powder was evenly spread inside sterilized Kraft envelopes and sealed in the presence of laminar flow, to maintain the sterilized conditions. The thickness of the envelope was maintained according to the space provided at input point of the instrument. The envelopes were exposed to different power levels and for variant residence time. All the treated fractions were checked for CFU of the organisms added and compared to one control sample where no microwave treatment was given. For standardization of processing protocol, single drug Hypericum perforatum was taken and then same treatments were repeated on different drugs as well.

### EXAMPLE 2

10 gms of herbal drug, Hypericum perforatum, was put inside the sterilized envelope and exposed to power level of 900 watts for a residence time of 5 minutes. One control sample was taken where no exposure was given. The microbial load when tested showed 98% decrease in bacterial lead.

### EXAMPLE 3

10 gms of drug Hypericum perforatum was put inside the sterilized envelope and exposed to power level of 800 watts for a residence time of 20 minutes. One control sample was taken where no exposure was given. The microbial load when tested showed 92% decrease in bacterial load.

### EXAMPLE 4

10 gms of herbal drug Hypericum perforatum was put inside the sterilized envelope and exposed to power level of 800 watts for a residence time of 30 minutes. One control sample was taken where no exposure was given. The microbial load when treated showed 97% decrease in bacterial load.

### EXAMPLE 5

10 gms of herbal drug Hypericum perforatum was put inside the sterilized envelope and exposed to power level of 900 watts for a residence time of 80 seconds. One control sample was taken where no exposure was given. The microbial load when tested showed 78% decrease in bacterial load.

### EXAMPLE 6

10 gms of herbal drug Hypericum perforatum was put inside the sterilized envelope and exposed to power level of 900 watts for a residence time of 80 seconds with gaps of 10 seconds. One control sample was taken where no exposure was given. The microbial load when tested showed 86% decrease in bacterial load.

Hence, Example 6 indicates that microbial load can be reduced to 86% when the substrate was exposed to 900 watts for a residence time of 80 seconds with the gap of 10 seconds with least air contamination.

### EXAMPLE 7

10 gms of herbal drug Piper longum was grounded and sieved through BS 30 mesh and spreaded inside the sterilized envelop. The drug was exposed to power level of 900 watts for a residence time of 80 seconds. One control sample was taken where no exposure was given. The microbial load when tested showed 75% decrease in bacterial load.

### EXAMPLE 8

10 gms of herbal drug Piper longum was grounded and sieved through BS 30 mesh and spreaded inside the sterilized envelop. The drug was exposed to power level of 900 watts for a residence time of 80 seconds with gaps of 10 seconds. The microbial load when tested showed 84% decrease in bacterial load.

### EXAMPLE 9

10 gms of herbal drug Withania somnifera was sieved through BS mesh 30 and spreaded inside the sterilized envelop. The drug was exposed to power level of 900 watts for a residence time of 80 seconds. The microbial load when tested showed 76% decrease in bacterial load.

### EXAMPLE 10

10 gms of herbal drug Withania somnifera was sieved through BS mesh 30 and spreaded inside the sterilized envelop. The drug was exposed to power level of 900 watts for a residence time of 80 seconds with gaps of 10 seconds. The microbial load when tested showed 83% decrease in bacterial load.

### Main Advantages of Present Invention

1. The equipment of present invention is simple, safe and continuous.

2. The equipment of the present invention is versatile, compact and can be operated by semi-skilled labour.

3. The present invention provides a single step process for simultaneously drying and sterilizing the herbal drugs.

4. The repeatability of the results is very high in the device of the present invention as compared to the conventional microwave machines.

5. The device requires minimum manual handling and gives high productivity of decontaminated products at less cost.

6. There is no change in the chemical composition of the material after sterilization.

7. The device of the present invention provides an entirely eco-friendly process for decontamination as no solvents and chemicals are involved.

8. The advantages associated with machine for the production of decontaminated products such as Medicinal Plant, spices will be obvious to persons skilled in the art.

1. A microbia decontaminator device for decontaminating herbal and/or pharmaceutical products, the said decontamination device comprising of:
a microwave oven provided with two slots, one on a front face and the other on a rear face of the microwave oven;
a conveyor belt passing through the microwave oven through the two slots and provided with a driving means for driving the conveyor belt;
said driving means being coupled to a computer which controls the driving means and in turn, the conveyor belt;
the assembly of the microwave oven, the conveyor belt and the driving means being covered by a shielding cage to prevent leakage of microwave radiation to atmosphere;
a feed hopper provided behind the rear face of the microwave oven and the conveyor belt for feeding the product to be decontaminated, and
an output chute being provided below the conveyor belt and in front of the front face of the microwave oven for collecting the decontaminated product.

2. A microbial decontaminator device as claimed in claim 1, wherein the microwave oven is a commercially available microwave oven operating at a power level in the range of 800 to 900 watts.

3. A microbial decontaminator device as claimed in claim 1, wherein a knob for controlling the status of the microwave oven and the operating power of the microwave oven is provided outside the shielding cage.

4. A microbial decontaminator device as claimed in claim 1, wherein the slots provided on the microwave oven are enough to let the conveyor to pass through.

5. A microbial decontaminator device as claimed in claim 1, wherein the capacity of the microwave oven is 28 liters.

6. A microbial decontaminator device as claimed in claim 1, wherein the conveyor belt is constructed of Teflon.

7. A microbial decontaminator device as claimed in claim 1, wherein the conveyor belt passes over two rollers of substantially same size.

8. A microbial decontaminator device as claimed in claim 7, wherein the rollers are coupled to the driving means through a coupling mechanism for driving the rollers.

9. A microbial decontaminator device as claimed in claim 7, wherein the rollers are provided with bearings for coupling to the driving means.

10. A microbial decontaminator device as claimed in claim 1, wherein the driving means is a motor.

11. A microbial decontaminator device as claimed in claim 1, wherein the motor is coupled to the computer using a parallel port or a LPT1 printer port.

12. A microbial decontaminator device as claimed in claim 1, wherein the time period for which the herbal or the pharmaceutical product is exposed to the microwave radiation is controlled by the computer.

13. A microbial decontaminator device as claimed in claim 1, wherein the shielding cage from a Faraday's cage thereby preventing leakage of microwave radiation to the atmosphere.

14. A microbial decontaminator device as claimed in claim 13, wherein the Faraday's cage is made up of M.S. Mesh and acrylic sheets.

15. A microbial decontaminator device as claimed in claim 13, wherein the Faraday's cage is made up of M.S. Mesh having 0.1 mm diameter holes and 6 mm thick acrylic sheet.

16. A microbial decontaminator device as claimed in claim 1, wherein the feeding hopper is provided such that a feeding end of the hopper is above the shielding cage and a discharge end is inside the shielding cage.

17. A microbial decontaminator device as claimed in claim 1, wherein the output chute is provided such that an output-collecting end is inside the shielding cage while an output discharge end is outside the shielding cage.
